(19) United States
(12) Patent Application Publication
(10) Pub. No.: US 2019/0064750 A1
(43) Pub. Date: Feb. 28, 2019

(54) PERSONAL PROTECTIVE EQUIPMENT AND METHODS OF MONITORING TIME OF USAGE OF PERSONAL PROTECTIVE EQUIPMENT

(71) Applicant: 3M INNOVATIVE PROPERTIES COMPANY, St. Paul, MN (US)

(72) Inventors: Steven T. AWISZUS, Woodbury, MN (US); Michael BAHNERS, Neuss (DE); Robert BIALLUCH, Willich (DE); Nadine E. BRUECKMANN, Düsseldorf (DE); Frank T. HERFORT, Korschenbroich (DE); Kiran S. KANUKURTHY, Cottage Grove, MN (US); Markus Gunther Wilfried LIERSE, Moenchengladbach (DE); Eric C. LOBNER, Woodbury, MN (US); Hugh Gerard Henry Tracey MURPHY, Lake Elmo, MN (US); Michael H. STALDER, Uedem (DE)

(73) Assignee: 3M INNOVATIVE PROPERTIES COMPANY, St. Paul, MN (US)

(21) Appl. No.: 15/757,021

(22) PCT Filed: Aug. 29, 2016
(86) PCT No.: PCT/US2016/049243

§ 371 Date: Mar. 2, 2018

Related U.S. Application Data
(60) Provisional application No. 62/214,433, filed on Sep. 4, 2015.

(51) Int. Cl. G04F 13/00; A42B 3/04; A61F 9/06; A61F 11/14; A41D 13/00; A62B 9/00; A62B 35/00; A62B 18/08; A43B 7/32; A43B 3/00

(52) U.S. Cl. CPC G04F 13/00; A42B 3/0466; A61F 9/06; A61F 11/14; A41D 13/0002; A41D 2600/202; A62B 35/0025; A62B 18/08; A43B 7/32; A43B 3/0005; A62B 9/006

(57) ABSTRACT

The present disclosure includes an article of PPE. The device includes a first sensor that detects whether the article of PPE is being worn by a user, a processing module and a communications module. The processing module includes a clock that measures the length of time that the article of PPE is being worn by the user, and memory for storage of usage data, wherein usage data includes the length of time the article of PPE has been worn by the user. The communications module is for wirelessly transmitting stored usage data to a device separate from the article of PPE.

### FIELD

The present description relates to personal protective equipment that is capable of detecting usage, including whether the device is being worn, logging usage data on the device and ultimately wirelessly transmitting usage data, and to a method of monitoring time of usage, including whether the device is being worn, of personal protective equipment.

### BACKGROUND

Maintaining the safety and health of workers is a major concern across many industries. Various rules and regulations have been developed to aid in addressing this concern. Such rules provide sets of requirements to ensure proper administration of personnel health and safety procedures. To help in maintaining worker safety and health, some individuals may be required to don, wear, carry, or otherwise use a personal protective equipment (PPE) article, if the individuals enter or remain in work environments that have hazardous or potentially hazardous conditions.

Known types of PPE articles include, without limitation, respiratory protection equipment (RPE), e.g., for normal condition use or emergency response; protective eyewear, such as visors, goggles, filters or shields; protective headwear, such as hard hats, hoods or helmets; hearing protection devices; protective shoes; protective gloves; other protective clothing, such as coveralls and aprons; protective articles, such as sensors, safety tools, detectors, global positioning devices, mining cap lamps and any other suitable gear.

One of the key determinations a worker must make with respect to a PPE is whether or not it remains functional and effective given the amount of use that it has undergone. Amount of use can include the amount of time the PPE was worn, whether the PPE was properly worn for that time period, the amount of time the PPE was powered or otherwise actively used, and the level of exposure in the environment to which the PPE is subjected. Improved methods for tracking, managing and using information related to these factors would be welcomed.

### SUMMARY

The present disclosure provides for, in part, an article of personal protective equipment (PPE) or an article of PPE that allows detection of whether the personal protective equipment is being worn by a user along with a clock that measures the length of time that the personal protective equipment is being worn by the user. Personal protective equipment is used interchangeably throughout. The present disclosure provides a variety of advantages over previously known solutions. For example, it allows a user to know when a device has been used for a length of time equivalent or equal to its maximum use time. The present disclosure allows detection of whether a worker is wearing PPE, and whether or not the worker or user is wearing the PPE continuously. The present disclosure allows employers to detect and track compliance of workers with regulations related to wearing PPE.

The present disclosure may provide additional advantages by allowing accurate tracking of the service life of the PPE. Further, the present disclosure may reduce cost of PPE expenditures and increase safety by allowing PPE to be replaced at the right time according to its service life, not prematurely or after the fact.

In one instance, the present disclosure includes an article of personal protective equipment (PPE). The device includes a first sensor that detects whether the article of PPE is being worn by a user, a processing module and a communications module. The processing module includes a clock that measures the length of time that the article of PPE is being worn by the user, and memory for storage of usage data, wherein usage data includes the length of time the article of PPE has been worn by the user. The communications module is for wirelessly transmitting stored usage data to a device separate from the article of PPE.

In another instance, the present disclosure includes an article of personal protective equipment (PPE), comprising a first sensor that detects whether the article of PPE is being worn by a user and an environmental sensor that detects an environmental factor. The article of PPE further includes a processing module and a communications module. The processing module includes a clock that measures the length of time that the article of PPE is being worn by the user, and memory for storage of usage data, wherein usage data includes the length of time the device has been worn by the user. The communications module for wirelessly transmitting stored usage data to a device separate from the article of PPE.

In some instances, the article of PPE is one of: a mask, a respirator, a hard hat, a welding helmet, protective hearing muffs, an eye protector, protective clothing, protective footwear or a fall protection harness.

In some instances, the first sensor is one of: an accelerometer, a capacitive sensor, a capacitive touch switch, a pressure switch, an acoustic sensor, a reed switch, a temperature sensor, a pressure sensitive switch, a gas sensor, a flow sensor and an optical sensor.

In some instances, the communications module includes at least one of: a radio frequency identification (RFID) tag, a ZigBee radio, a Bluetooth transmitter, an ANT node and a Wi-Fi device.

In some instances, the article of PPE further comprises a sealing membrane in contact with the skin of the user when the user is wearing the article of PPE, wherein the sensor is disposed in the sealing membrane in order to sense contact of the sealing membrane with the skin of the user.

In some instances, the article of PPE comprises a second sensor, wherein the second sensor detects whether the article of PPE is in active use.

In some instances, the article of PPE is a powered device, and the processor disables the device's ability to power “ON” if the device is not being worn.

In some instances, the second sensor detects whether the article of PPE is in active use by detecting at least one of: the article of PPE being powered “ON”, or actuation or movement of a component of the article of PPE relative to the remainder of the article of PPE.

In some instances, the usage data further comprises the length of time the article of PPE is in active use.

In some instances, the processor uses the usage data to determine necessary servicing or end-of-life of the article of PPE.

In some instances, the article of PPE is a powered device, and the processor disables the device's ability to power “ON” upon determining necessary servicing or end-of-life of the article of PPE.

In some instances, the environmental sensor detects at least one of the following environmental characteristics: air quality and ambient noise levels.

In some instances, the environmental sensor generates an environmental rating based on the detected environmental characteristics.

In some instances, the usage data further includes the environmental rating.

In some instances, the processor uses the usage data to determine necessary servicing or end-of-life of the article of PPE.

In some instances, the article of PPE is a powered device, and wherein the processor disables the device's ability to power “ON” upon determining necessary servicing or end-of-life of the article of PPE.

### BRIEF DESCRIPTION OF THE DRAWINGS

The following figures provide illustrations of the present invention. They are intended to further describe and clarify the invention, but not to limit scope of the invention.

FIG. 1 is a block diagram of components of an article of PPE with a sensor to detect whether the article of PPE is being worn.

FIG. 2 is a block diagram of components of an article of PPE with a sensor to detect whether the article of PPE is being worn and an environmental sensor.

FIGS. 3A and 3B are views of a sealing frame of an exemplary article of PPE and a pressure switch, respectively.

FIG. 4 is a cross-sectional view of a reed switch.

FIG. 5 is a cross-sectional view of an exemplary sensor, a capacitive touch switch.

FIG. 6 is a flow chart related to the operation of an article of PPE consistent with the present description.

The figures are not necessarily to scale. Like numbers used in the figures refer to like components. However, it will be understood that the use of a number to refer to a component in a given figure is not intended to limit the component in another figure labeled with the same number.

### DETAILED DESCRIPTION

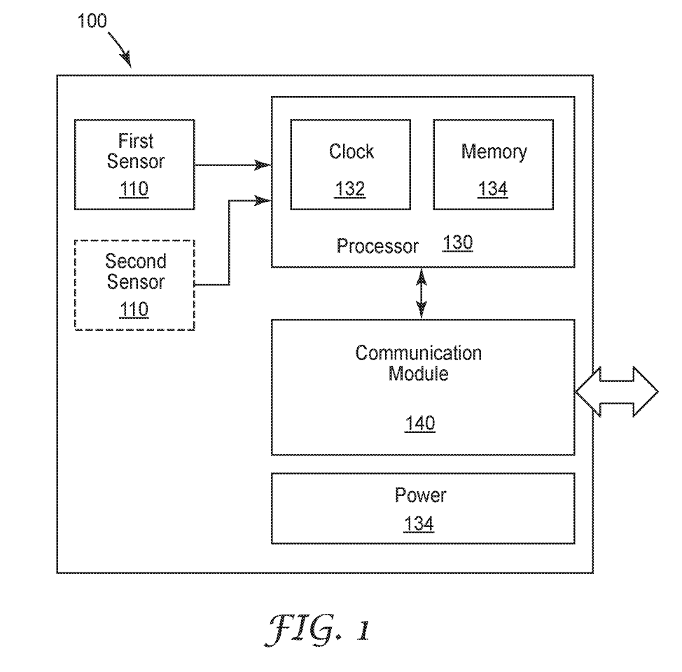

FIG. 1 is a block diagram of components of an article of PPE with a sensor to detect whether the article of PPE is being worn. The components shown in FIG. 1 are exemplary, and not intended to be limiting. Other combinations of components and variations on the components shown are within the scope of the present disclosure. FIG. 1 includes article of PPE 100. Article of PPE 100 may be any type of article of PPE. Examples of article of PPE include without limitation, respiratory protection equipment (RPE), e.g., for normal condition use or emergency response; protective eyewear, such as visors, goggles, filters or shields; protective headwear, such as hard hats, hoods or helmets; hearing protection devices; protective shoes; protective gloves; other protective clothing, such as coveralls and aprons; protective articles, such as sensors, safety tools, detectors, global positioning devices, mining cap lamps and any other suitable gear. Article of PPE 100 includes at least one sensor and several other electronic components. Article of PPE 100 includes first sensor 110. Article of PPE 100 may optionally include second sensor 120. Article of PPE 100 further includes processor 130, including clock 132 and memory 134. Article of PPE also includes communication module 140 and power module 160. While the various components or modules are shown as separate in FIG. 1, they may be integrated into any combination of electronic components and sensors consistent with the present disclosure.

First sensor 110 is secured to or otherwise integrated into article of PPE 100. For example, first sensor 110 may be adhered to, embedded in, manufactured as part of, fastened, sewn, friction fitted, mechanically clipped, welded (e.g. ultrasonically) or molded etc. onto or into article of PPE 100 or any component thereof. First sensor 110 detects whether the article of PPE is being worn by a user. First sensor 110 can be a variety of types of sensors. For example, first sensor 110 may be an accelerometer, a capacitive sensor, a capacitive touch switch, a pressure switch, an acoustic sensor, a reed switch, a temperature sensor, a pressure sensitive switch, a gas sensor, a flow sensor or an optical sensor. First sensor 110 may be any other sensor that detects whether article of PPE is being worn by a user. First sensor 110 may detect whether article of PPE is being worn by user in a variety of ways, including detecting pressure, breath, movement, capacitance from skin, noise or sound and temperature differential. Other ways to detect whether article of PPE is being worn by user are within the scope of the present disclosure and will be apparent to those of skill in the art upon reading the present disclosure.

In some instances, article of PPE 100 may include second sensor 120. Second sensor 120 detects whether article of PPE 100 is in active use. Active use includes use of the article of PPE that would decrease the total length of time required until replacement of disposable parts of the article of PPE, required service time, or end of life of the article of PPE. Active use could include exposure to particular elements, power supplied to the device or electronic or other triggered response to exposure. Second sensor 120 may be a variety of sensors such as accelerometer, a capacitive sensor, a capacitive touch switch, a pressure switch, an acoustic sensor, a reed switch, a temperature sensor, a pressure sensitive switch, a gas sensor, a flow sensor, a galvanic skin resistance sensor or an optical sensor. Second sensor 120 detects whether the article of PPE is in active use by detecting at least one of: the article of PPE being powered “ON”, or actuation or movement of a component of the article of PPE relative to other components of the article of PPE (for example, movement of an exhalation valve on a respirator), noise, air quality, temperature, air flow, exposure to light, chemical or environmental exposure.

Article of PPE 100 further includes processing module 130. Processing module 130 includes at least a clock 132 and memory 134. Clock 132 measures the length of time that the article of PPE is being worn by the user. Memory 134 is for storage of usage data, wherein usage data includes the length of time the article of PPE 100 has been worn by the user.

Processing module 130 may be an integral part of first sensor 110 or may be a separate component with an electrical connection to first sensor 110. Processing module 130 may be any type of processing or computing device as will be apparent to one of skill in the art upon reading the present disclosure. For example, processing module may be a microcontroller, FPGA or other computing device as will be apparent to one of skill in the art upon reading the present disclosure.

Article of PPE 100 further includes communication module 140 for wirelessly transmitting stored usage data to a device separate from the article of PPE 100. In some configurations, the communications module 140 includes at least one of: a radio frequency identification (RFID) tag (active or passive), a ZigBee radio, a Bluetooth transmitter, an ANT radio, and a Wi-Fi device. Communications module 140 may include any other type of communications device, as will be apparent to one of skill in the art upon reading the present disclosure.

Power module 160 provides power to other electronic components in article of PPE 100, including, for example, first sensor 110, second sensor 120, processor 130, clock 132, memory 134 and communication module 140. Power module 160 may use a variety of power sources, such as a battery or other stored power. Power module may also use energy harvesting, photovoltaic energy, thermoelectric energy, vibratory energy, piezoelectric energy, etc.

Each of the components shown in FIG. 1 can be in communication with each other, and can be configured in a variety of manners, as will be apparent to one of skill in the art upon reading the present disclosure. For example, first sensor 110 and second sensor 120 may be powered by power module 160, be configured to transmit data to processor 130, and may also be configured to receive data from processor 130. In some instances, first sensor 110 and second sensor 120 may communicate directly with each other. First sensor 110, second sensor 120, processor 130, clock 132 and memory 134 may all work together to calculate and store usage data. Usage data includes information related to a user's wear of the article of PPE, use of the article of PPE, and/or information about the environment the article of PPE is used in or stored in. Specifically, usage data can include the length of time the article of PPE 100 has been worn by the user, the length of time the article of PPE 100 is in active use. Usage data may be used to calculate a variety of device-specific information such as time left until component replacement or device service is required or time left until device end of life. In some instances, the processor may use the usage data to determine necessary servicing or end-of-life of the article of PPE. In some instances, the article of PPE is a powered device, and wherein the processor disables the device's ability to power “ON” upon determining necessary servicing or end-of-life of the article of PPE.

While the various components or modules are shown as separate in FIG. 1, they may be integrated into any combination of electronic components and sensors consistent with the present disclosure.

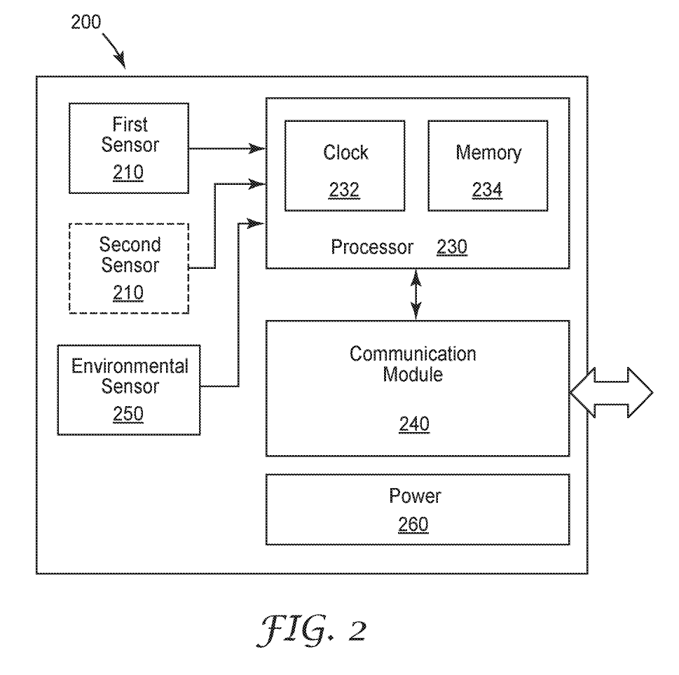

FIG. 2 is a block diagram of components of an article of PPE with a sensor to detect whether the article of PPE is being worn and an environmental sensor. Similar to FIG. 1, FIG. 2 is a block diagram of components of an article of PPE with a sensor to detect whether the article of PPE is being worn. The components shown in FIG. 2 are exemplary, and not intended to be limiting. Other combinations of components and variations on the components shown are within the scope of the present disclosure.

Article of PPE 200 may be any type of article of PPE. Examples of article of PPE include without limitation, respiratory protection equipment (RPE), e.g., for normal condition use or emergency response; protective eyewear, such as visors, goggles, filters or shields; protective headwear, such as hard hats, hoods or helmets; hearing protection devices; protective shoes; protective gloves; other protective clothing, such as coveralls and aprons; protective articles, such as sensors, safety tools, detectors, global positioning devices, mining cap lamps and any other suitable gear. Article of PPE 200 includes at least one sensor and several other electronic components. Article of PPE 200 includes first sensor 210. Article of PPE 200 may optionally include second sensor 220. Article of PPE 200 includes environmental sensor 250. Article of PPE 200 further includes processor 230, including clock 232 and memory 234. Article of PPE also includes communication module 240.

Each of first sensor 210, second sensor 220, processor 230, clock 232, memory 234, communication module 240 and power module 260 have at least the same functionality as the parallel component shown in FIG. 1. Each of these components in FIG. 2 can also be any of the specific components for the parallel component as described with respect to FIG. 1.

Environmental sensor 250 detects an environmental factor. An environmental factor can be anything, whether biotic or abiotic, that influences performance, degradation or other aspects of an article of PPE, or would otherwise negatively impact a user of the article of PPE. Examples of environmental factors include: air quality, temperature, ambient noise levels, presence of contaminants or chemicals, light, obstacles or potential or actual impact with another object. Environmental sensor may be any of a variety of sensors, including, for example, a thermometer, accelerometer, barometer, gas sensor, flow sensor, an acoustic sensor, an optical sensor or any other sensor as will be apparent to one of skill in the art upon reading the present disclosure.

Environmental sensor may also detect an environmental factor by receiving information about an environmental factor from a wireless communication device in a location known to have certain environmental factors. For example, a Bluetooth transmitter, such as one communicating using the iBeacon protocol, or an Estimote sold by Estimote, Inc. of New York, N.Y. could be programmed with a profile for the location the transmitter is disposed in. A location profile may include information related to the location, including any environmental factor as described herein. A location profile may also include information about emergency situations, number of articles of PPE the number of users in an area. The location profile information can be transmitted by the wireless communication device in the location and may be received by either the environmental sensor 250 or the communications module 240 in the article of PPE 200.

In some instances, environmental sensor may generate an environmental rating based on an environmental factor or multiple environmental factors. An environmental rating may be a scalar value or any other assigned number, letter, color or other type of rating used to classify the environmental factor. For example an environmental rating could be determined based on the comparison of various hazard levels with pre-determined benchmarks for those particular hazards. In some instances, if a single factor has a high hazard level, the environmental rating may be designated to indicate “danger”. In some multiple high hazard levels it may be required to rate an environment as dangerous, or as having a high hazard rating.

Processor 230 can use information about the length of time the article of PPE has been worn and information related to environmental factors, including for example, and environmental rating, to determine remaining service life, necessary servicing or end-of-life for the article of PPE or a component.

In some instances, where the article of PPE is a powered device, the processor the processor disables the device's ability to power “ON” upon determining necessary servicing or end-of-life of the article of PPE.

In some instances, processor 230 may also use information from second sensor 220, such as the length of the time that the article of PPE is in active use, in combination with other information such as other types of usage data to determine remaining service for the article of PPE.

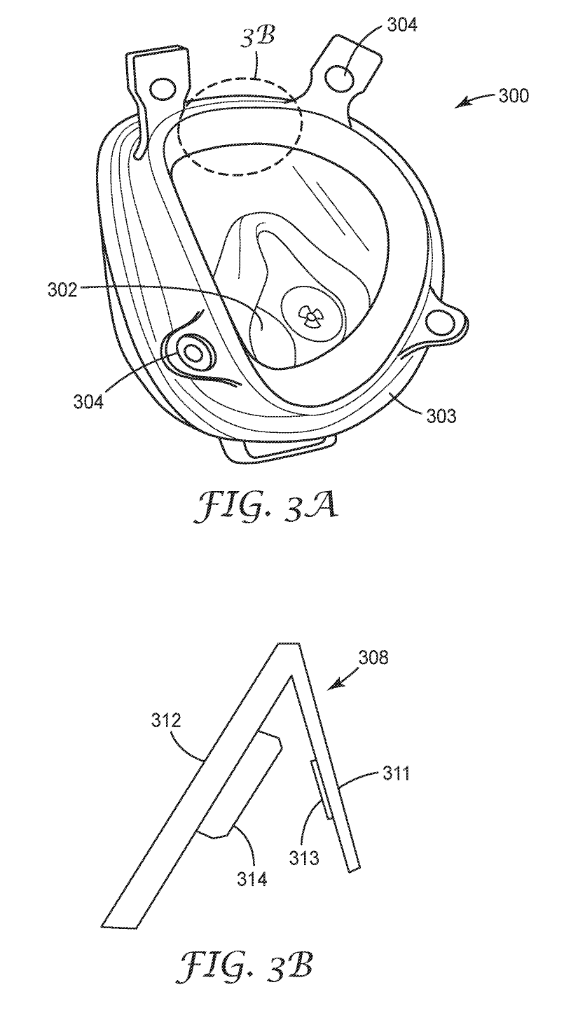

FIGS. 3A and 3B are views of a sealing frame of an exemplary article of PPE and a pressure switch, respectively. FIG. 3A shows an article of PPE, specifically, half face respirator 300. Respirator 300 includes sealing membrane 303, exhalation valve 302 and strap receptacles 304. Strap receptacles 304 are used to secure rubber or other straps to the respirator, such straps being designed to hold respirator 300 in contact with a face of a user. Sealing membrane 303 seals around the face of the user, such that at least the nose and mouth of the user are enclosed by respirator 300. One example of a respirator consistent with the present disclosure is the 3M™ Half Facepiece Reusable Respirator 6200, sold by 3M Company of St. Paul, Minn.

Respirator 300 includes a pressure switch, such as the one shown in FIG. 3B, disposed between folds of or otherwise in the sealing membrane. Pressure switch 308 is activated when the respirator 300 is worn by the pressure on sealing membrane 303 created by the respirator 300 being secured around a user's heads by straps or another attachment mechanism. Sensor 308 is disposed in the sealing membrane 303 in order to sense contact of the sealing membrane with the skin of the user. Other types of sensors may be used instead of pressure sensor to detect when a user is wearing respirator 300. For example, a capacitive sensor may be used to detect contact between the sealing membrane and a user's face. Other types of sensors may be used, as will be apparent to one of skill in the art upon reading the present disclosure.

Pressure sensor 308 or another sensor can be in communication with a processor (not shown in FIG. 3A) or other electronic devices or sensors and can transmit information to the processor indicating whether the respirator 300 is being worn to form part of the usage data for the respirator 300.

FIG. 3B shows a portion of pressure sensor 308, such as one that could be secured to or part of respirator 300 shown in FIG. 3A. Pressure sensor 308 includes two arms 311, 312, and a conductive element 313, 314 on each arm 311, 312. Pressure sensor 308 can be disposed in a respirator so that when the sealing membrane is in contact with a user's face, the pressure between the frame of the respirator and the user's face forces the arms 311, 312 of pressure sensor 308 toward each other such that conductive elements 313 and 314 come into contact with each other. When conductive elements 313 and 314 come into contact with each other, they complete a circuit such that the pressure switch 308 is activated or actuated and transmits information indicating that it is activated to a processor. The processor correlates actuation of the pressure switch 308 with respirator wear time. Similarly, the processor correlates de-actuation or non-actuation of the pressure switch 308 with non-wear of the respirator or article of PPE.

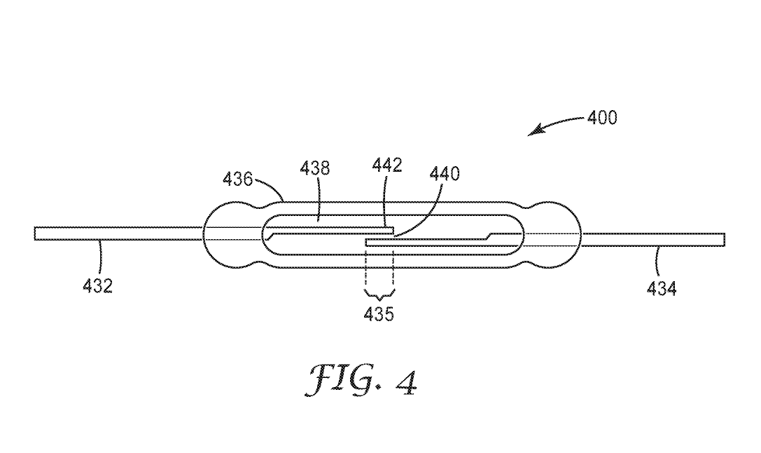

FIG. 4 is a cross-sectional view of a reed switch 400. Reed switch 400 is another example of a sensor that could be used in combination with a respirator or any other type of article of PPE to determine whether the article of PPE is being worn by a user. A variety of reed switches can be used. One example of a reed switch is described in U.S. Pat. No. 2,264,746 to Ellwood, incorporated herein by reference.

Reed switch 400 is an electrical switch that is activated by an applied magnetic field in proximity to the switch. Reed switch 400 include a first reed blade 432 and a second reed blade 434 that overlap one another at region 435 within a glass capsule or other type of capsule. Within the glass capsule is an inert gas 438 that surrounds the first and second reed blades. Illustrated also in FIG. 4 is the contact gap between the two reeds 440, as well as the contact plating 442 on the overlap portion of the two reeds. The reed switch 400 can be actuated by bringing a magnet or magnetic field near the switch. Once the magnetic field is moved away from the reed switch, it returns to its original position. Reed switch 400 can incorporated into or installed in an article of PPE such that when the PPE is worn by a user, a piece of magnetic material attached to the article of PPE is brought into proximity of the reed switch 400 such that the reed switch 400 will be activated.

Reed switch 400 can be connected to a processor and can transmit information indicating that it is actuated to a processor. The processor correlates actuation of the reed switch 400 with respirator or article of PPE wear time. Similarly, the processor correlates de-actuation or non-actuation of the reed switch 400 with non-wear of the respirator or article of PPE to calculate the length of time the article of PPE is worn.

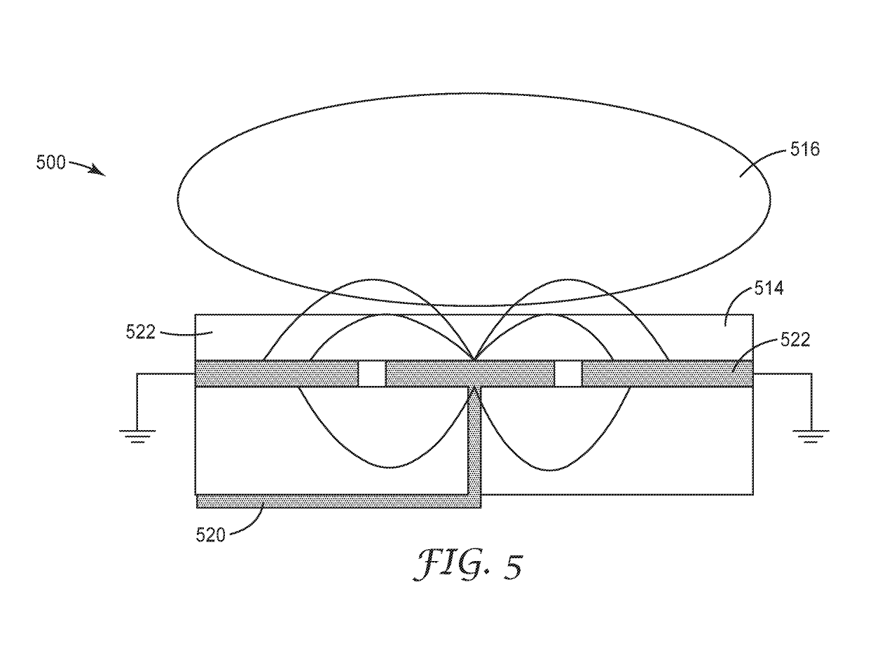

FIG. 5 is a cross-sectional view of an exemplary sensor that could be used consistent with the present disclosure, a capacitive touch switch 500. Capacitive touch switch 500 requires only one electrode to function. It operates by detecting body capacitance. As shown in FIG. 5, electrode 520, having copper traces 522 may be positioned behind a non-conductive panel 514. Non-conductive panel may be made up of any appropriate non-conductive material, potentially one that offers structural support, such as wood, glass or plastic. The capacitive touch switch detects body capacitance from skin 516 of the user when in close proximity to the skin of user. Capacitive touch switch 500 can be disposed on or otherwise secured to an article of PPE such that it only comes into sufficiently close proximity to the skin of the user to be actuated when the user is wearing the article of PPE.

Capacitive touch switch can be connected to or otherwise communicate with a processor and transmit information indicating it is actuated to a processor. The processor correlates actuation of the capacitive touch switch 500 with respirator or article of PPE wear time. Similarly, the processor correlates de-actuation or non-actuation of the capacitive touch switch 500 with non-wear of the respirator or article of PPE to calculate the length of time the article of PPE is worn.

Other types of sensors, including those discussed throughout the present disclosure and those that will be apparent to one of skill in the art upon reading the present disclosure, may be used consistent with the scope of the present disclosure.

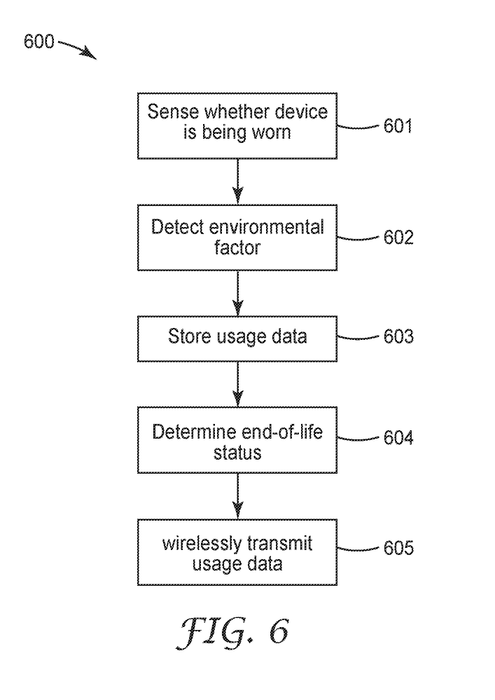

FIG. 6 is a flow chart 600 related to the operation of an article of PPE consistent with the present description. The steps shown in flow chart 600 are exemplary, and other steps may also be taken, or some steps shown in flow chart 600 may not be taken, consistent with the present disclosure. Further, the steps shown in flow chart 600 may be taken in an order and be consistent with the scope of the present disclosure. The order shown in FIG. 6 is one potential configuration within the scope of the present disclosure.

The steps shown in flow chart 600 may be performed by an article of PPE and any sensors or electronic components in the article of PPE or in communication with the article of PPE.

In step 601, a first sensor in an article of PPE detects whether the device is being worn. The first sensor can be a variety of types of sensors. For example, the first sensor may be an accelerometer, a capacitive sensor, a capacitive touch switch, a pressure switch, an acoustic sensor, a reed switch, a temperature sensor, a pressure sensitive switch, a gas sensor, a flow sensor or an optical sensor. The first sensor may be any other sensor that detects whether article of PPE is being worn by a user. The first sensor may detect whether article of PPE is being worn by user in a variety of ways, including detecting pressure, breath, movement, capacitance from skin, noise or sound and temperature differential. Other ways to detect whether article of PPE is being worn by user are within the scope of the present disclosure and will be apparent to those of skill in the art upon reading the present disclosure.

In step 602, an environmental sensor detects an environmental factor. An environmental factor can be anything, whether biotic or abiotic, that influences living organisms. Examples of environmental factors include: air quality, temperature, ambient noise levels, presence of contaminants or chemicals, light, obstacles or potential or actual impact with another object. Environmental sensor may be any of a variety of sensors, including, for example, a thermometer, accelerometer, barometer, gas sensor, flow sensor, an acoustic sensor, an optical sensor or any other sensor as will be apparent to one of skill in the art upon reading the present disclosure. Environmental sensor can detect an environmental factor in any of a variety of manners, as described throughout the present disclosure.

While not shown in flow chart 600, in some instances a second sensor may detect whether the article of PPE is in active use. The second sensor may detect whether the article of PPE is in active use by detecting at least one of: the article of PPE being powered “ON”, or actuation or movement of a component of the article of PPE relative to the remainder of the article of PPE.

In step 603, usage data is stored. Usage data can be stored by a processor module including memory in the article of PPE. Usage data includes the length of time the article of PPE has been worn by the user. Usage data may also include the length of time the article of PPE has been in active use. Usage data can include a variety of types of information related to a user's wear and use of the article of PPE, the environment the article of PPE is used and stored it and other information relating to use of the article of PPE.

In step 604, the processor determines end-of-life status of the article of PPE. The processor may use the usage data to determine necessary servicing or end-of-life of the article of PPE. The processor may use information such as usage data, including the length of time the article of PPE is worn, the length of time the article of PPE is actively used, and information about the environment the article of PPE is used and stored in to determine necessary servicing or end-of-life of the article of PPE. In some instances, the article of PPE is a powered device, and wherein the processor disables the device's ability to power “ON” upon determining necessary servicing or end-of-life of the article of PPE.

In step 605, the communication module wirelessly transmits usage data to or other information, such as end-of-life or necessary servicing status to a device separate from the article of PPE. This separate device or system could include a database or other system for storing and managing information about the article of PPE.

Other steps in the process described will be apparent to one of skill in the art upon reading the present disclosure, and are intended to be within the scope of the present disclosure. The steps illustrated and described are exemplary and not intended to be exhaustive.

### PROPHETIC EXAMPLES

### Example 1: Capacitive Sensor in Respiratory or Hearing PPE

A capacitive electrode sensor (or capacitive sensor), for example, an AT42QT4120 touch sensor chip available from Atmel Corporation, headquartered in San Jose, Calif., can be positioned inside the nose area of an article of PPE (PPE) such as in a respirator cup of a disposable respirator, for example 3M 8210 from 3M Company, St. Paul, Minn.; or inside the liner of a hearing protector muff device, for example 3M™ Peltor™ Optime™105 Earmuff, from 3M Company, St. Paul, Minn.; or inside the liner of the cup of a hearing protector communication headset, such as 3M™ Peltor™ WS™ 5 Headset from 3M Company, St. Paul, Minn. The capacitive electrode sensor may be used to sense the resistance of the skin when the PPD comes into contact with the skin of the wearer. This capacitive sensor can be paired with a wireless microcontroller for gathering periodic and/or real time data that can be stored and forwarded later or sent over the wireless channel in real time to a device separate from the PPD, such as a central database. Real time data may be taken from a log recording resistance measurements over time. The data from the capacitive electrode sensor may be sent to a central database and documented. These data readings can be processed by the conditions such as the cumulative time that the PPD is worn during a predetermined criteria, such as length of the wearer's work shift.

### Example 2: Multiple Capacitive Sensors in Respiratory PPE

Multiple wireless capacitive electrode sensors can be distributed throughout for example, the face seal area of an elastomeric full face-piece respirator such as 6000 Full Face, of 3M Company, St. Paul, Minn. The capacitive electrode sensors can be placed at specific contact areas near, for example, the nose, cheek bones, and jaw areas. The capacitive electrode sensors may be used to sense the resistance of the skin over time. Real time data may be taken from a log recording resistance measurements over time. The data from the capacitive electrode sensors may be sent to a central database and documented. Changes in the data from the capacitive electrode sensors over the interval during which a respirator is worn can be used as an indicator of fit, or protection from the wearer's exposure to the work environment. By monitoring the resistance data over a period of days, a determination may be made that a worker is not adequately protected within the working environment, and efforts undertaken by appropriate personnel to correct the matter, such as reassessing the fit of the respirator, refitting to a different type or size of respirator, or providing additional training in the proper donning of the respirator. Data related to fit is an example of a type of usage data. Data from the capacitive electrode sensors can be used to determine the length of time the respirator is worn.

### Example 3: Temperature and/or Humidity Sensors in Respiratory PPE

A temperature sensor, for example, a CHAL-010-BW thermocouple available from Omega Engineering, Inc. of Stamford, Conn., can be positioned for example, inside the article of PPE in the exhalation flow path, or the path of air flow exhaled from a user's mouth, of an elastomeric full face-piece respirator such as the 6000 Full Face, of 3M Company, St. Paul, Minn. The Omega CHAL-010-BW fine gauge unsheathed small thermal mass thermocouple sensor can be used for this application at least in part because of its small thermal mass. Such small mass thermal sensors can respond to changes within a small time period, less than 2 seconds, or to cyclical changes in temperature of about 1 degree Celsius, as typically occur during a wearer's inhalation and exhalation cycle. Changes in temperature over a specified time interval can be used as an indicator of the length of time the respirator is being worn. Changes in temperature over a specified time interval can also be used as an indicator of the length of time the respirator is being actively used. These data readings from the temperature sensor can be used to determine the length of time the respirator is being worn during a predetermined criteria such as a required time interval, such as the wearer's work shift. It is to be noted that, NTC thermistors can also be used in place of thermocouples. One such example is USP10982 available from U.S. Sensor Corp. of Orange, Calif.

Alternatively a relative humidity (RH) sensor, for example, a Si7023-A20 humidity and temperature sensor chip from Silicon Laboratories, Inc. of Austin, Tex., can be positioned, for example, in the path of air flow or air exhaled from the user's mouth of an elastomeric full face-piece respirator such as 6000 Full Face, of 3M Company, St. Paul, Minn. Similarly, the humidity sensor would be of a fast response type that would respond quickly to changes within a small time period, 5 to 10 seconds. Humidity values can be as high as 85% inside a respirator mask, even when the environmental humidity is much lower. Changes in relative humidity over a specified time interval during which a respirator is worn can be used as an indicator of the length of time the respirator is worn, or the length of time the respirator is in active use. These data readings from the humidity sensor can be used to determine the length of time the respirator is being worn during a predetermined criteria such as a required time interval, such as the wearer's work shift.

The temperature and RH sensors can be used in combination such as in the form of a combined sensor Si7023-A20 temperature and RH sensor chip in a small form factor. This would provide the advantage of detecting human breath along with cyclical temperature variations to determine the length of time the device is worn or is actively used with a higher accuracy than just using either a temperature or a RH sensor.

### Example 4: Two or More Temperature Sensors in Respiratory PPE

In addition to the temperature sensor described in Example 3, a second temperature sensor can be positioned on an outer surface of the article of PPE to monitor the ambient or environmental temperature. A differential reading between the internal and external sensors can be used to determine if the PPE is being worn. More specifically, when the worker is wearing the PPE, internal temperature detected by the internal sensor will both differ from the temperature detected by the external sensor and will exhibit a pattern corresponding to human breathing. When the worker is not wearing the PPE, the temperature detected by the internal and external temperature sensors will be the same, or within a given range of each other. The temperature data detected by the internal and external temperature sensor can be used to determine the length of time the device is worn and/the length of time the device is in active use.

### Example 5: Acoustic Sensor in Respiratory PPE

An acoustic circuit, including for example, a standard piezo microphone such as a Panasonic WM-61A from Panasonic Corporation, of Kadoma, Osaka Prefecture, in combination with a low noise pre-amplifier such as the OPA371 from Texas Instruments, of Dallas, Tex., can be used with necessary filters and frequency tuning characteristics as will be understood by one of skill in the art upon reading the present disclosure, to isolate to human breathing frequencies. The acoustic circuit can be positioned, for example, in the exhalation flow path of an elastomeric full face-piece respirator such as 6000 Full Face, of 3M Company, St. Paul, Minn. Real time data may be taken from a log recording acoustic sound level measurements over time. The data from the acoustic circuit may be stored locally and/or may be sent to a central database and documented. These acoustic data readings can be used to determine the length of time the device is worn and/the length of time the device is in active use.

### Example 6: Acoustic Sensor in Hearing PPE

An acoustic circuit, for example, a standard piezo microphone such as a Panasonic WM-61A from Panasonic Corporation of Newark, N.J., in combination with a low noise pre-amplifier such as OPA371 from Atmel Corporation of Burnsville, Minn., can be used with necessary filters and frequency tuning characteristics monitor use of hearing protection equipment such as hearing protector muff device, for example 3M™ Peltor™ Optime™ 105 Earmuff, from 3M Company, St. Paul, Minn.; or inside the liner of the cup of a Hearing Protector Communication Headset, such as 3M™ Peltor™ WS™ 5 Headset from 3M Company, St. Paul, Minn.

### Example 7: Active Wireless Device Integration

The PPE and sensors of Examples 1-6 can further be coupled with a communication module including active wireless device integrated into or otherwise secured to the PPE, such as NRF51822 Bluetooth Low Energy (BLE) chip, from Nordic Semiconductor of Oslo, Norway. The BLE chip can receive signals from a network Bluetooth transmitters or beacons in the area, and can use information in the received signals to determine the location of the user within a working environment. A processor in the PPE can determine whether the user was wearing the PPE during a time that the user was in a hazardous environment, but using data from the Bluetooth beacons in the environment in combination with sensors to determine the length of time the PPE is worn. This data can be used to determine compliance with PPE usage requirements. For example, a high hazard area would require strict compliance with the worker not allowed to remove the PPE even for a small time interval. In contrast, the worker may be allowed to remove the PPE in a hazard free area. The device described herein can monitor compliance with both of these scenarios.

### Example 8: Sending Usage Data to a Central Database

A communication module in the PPE described in Examples 1-8 sends usage data, including the length of time the device has been worn by the user and the length of time the personal protective equipment is in active use, to a central database. The central database can produce reports indicating the cumulative time that the PPD was worn during a predetermined time interval or over the life of the device. The central database uses the usage data to determine necessary servicing or end-of-life of the article of PPE.

It will be appreciated that numerous and varied other arrangements may be readily devised by those skilled in the art without departing from the spirit and scope of the invention as claimed.

It will be appreciated that based on the above description, aspects of the disclosure include methods and systems for determining time of use (wear time) of articles, such as PPE articles, by determining if they satisfy at least one criterion.

Although the methods and systems of the present disclosure have been described with reference to specific exemplary embodiments, those of ordinary skill in the art will readily appreciate that changes and modifications may be made thereto without departing from the spirit and scope of the present disclosure.

In the present detailed description of the preferred embodiments, reference is made to the accompanying drawings, which illustrate specific embodiments in which the invention may be practiced. The illustrated embodiments are not intended to be exhaustive of all embodiments according to the invention. It is to be understood that other embodiments may be utilized and structural or logical changes may be made without departing from the scope of the present invention. The following detailed description, therefore, is not to be taken in a limiting sense, and the scope of the present invention is defined by the appended claims.

Unless otherwise indicated, all numbers expressing feature sizes, amounts, and physical properties used in the specification and claims are to be understood as being modified in all instances by the term “about.” Accordingly, unless indicated to the contrary, the numerical parameters set forth in the foregoing specification and attached claims are approximations that can vary depending upon the desired properties sought to be obtained by those skilled in the art utilizing the teachings disclosed herein.

As used in this specification and the appended claims, the singular forms “a,” “an,” and “the” encompass embodiments having plural referents, unless the content clearly dictates otherwise. As used in this specification and the appended claims, the term “or” is generally employed in its sense including “and/or” unless the content clearly dictates otherwise.

Spatially related terms, including but not limited to, “proximate,” “distal,” “lower,” “upper,” “beneath,” “below,” “above,” and “on top,” if used herein, are utilized for ease of description to describe spatial relationships of an element(s) to another. Such spatially related terms encompass different orientations of the device in use or operation in addition to the particular orientations depicted in the figures and described herein. For example, if an object depicted in the figures is turned over or flipped over, portions previously described as below or beneath other elements would then be above or on top of those other elements.

As used herein, when an element, component, or layer for example is described as forming a “coincident interface” with, or being “on,” “connected to,” “coupled with,” “stacked on” or “in contact with” another element, component, or layer, it can be directly on, directly connected to, directly coupled with, directly stacked on, in direct contact with, or intervening elements, components or layers may be on, connected, coupled or in contact with the particular element, component, or layer, for example. When an element, component, or layer for example is referred to as being “directly on,” “directly connected to,” “directly coupled with,” or “directly in contact with” another element, there are no intervening elements, components or layers for example. The techniques of this disclosure may be implemented in a wide variety of computer devices, such as servers, laptop computers, desktop computers, notebook computers, tablet computers, hand-held computers, smart phones, and the like. Any components, modules or units have been described to emphasize functional aspects and do not necessarily require realization by different hardware units. The techniques described herein may also be implemented in hardware, software, firmware, or any combination thereof. Any features described as modules, units or components may be implemented together in an integrated logic device or separately as discrete but interoperable logic devices. In some cases, various features may be implemented as an integrated circuit device, such as an integrated circuit chip or chipset. Additionally, although a number of distinct modules have been described throughout this description, many of which perform unique functions, all the functions of all of the modules may be combined into a single module, or even split into further additional modules. The modules described herein are only exemplary and have been described as such for better ease of understanding.

If implemented in software, the techniques may be realized at least in part by a computer-readable medium comprising instructions that, when executed in a processor, performs one or more of the methods described above. The computer-readable medium may comprise a tangible computer-readable storage medium and may form part of a computer program product, which may include packaging materials. The computer-readable storage medium may comprise random access memory (RAM) such as synchronous dynamic random access memory (SDRAM), read-only memory (ROM), non-volatile random access memory (NVRAM), electrically erasable programmable read-only memory (EEPROM), FLASH memory, magnetic or optical data storage media, and the like. The computer-readable storage medium may also comprise a non-volatile storage device, such as a hard-disk, magnetic tape, a compact disk (CD), digital versatile disk (DVD), Blu-ray disk, holographic data storage media, or other non-volatile storage device.

The term “processor,” as used herein may refer to any of the foregoing structure or any other structure suitable for implementation of the techniques described herein. In addition, in some aspects, the functionality described herein may be provided within dedicated software modules or hardware modules configured for performing the techniques of this disclosure. Even if implemented in software, the techniques may use hardware such as a processor to execute the software, and a memory to store the software. In any such cases, the computers described herein may define a specific machine that is capable of executing the specific functions described herein. Also, the techniques could be fully implemented in one or more circuits or logic elements, which could also be considered a processor.

1. An article of personal protective equipment (PPE), comprising:
a first sensor that detects whether the article of PPE is being worn by a user;
a processing module including:
a clock that measures the length of time that the article of PPE is being worn by the user, and
memory for storage of usage data, wherein usage data includes the length of time the article of PPE has been worn by the user; and
a communications module for wirelessly transmitting stored usage data to a device separate from the article of PPE.

2. The article of PPE of claim 1, wherein the article of PPE is one of: a mask, a respirator, a hard hat, a welding helmet, protective hearing muffs, an eye protector, protective clothing, safety footwear or a fall protection harness.

3. The article of PPE of claim 1, wherein the first sensor is one of: an accelerometer, a capacitive sensor, a capacitive touch switch, a pressure switch, an acoustic sensor, a reed switch, a temperature sensor, a pressure sensitive switch, a gas sensor, a flow sensor and an optical sensor.

4. (canceled)

5. The article of PPE of claim 1, further comprising a sealing membrane in contact with the skin of the user when the user is wearing the article of PPE, wherein the sensor is disposed in the sealing membrane in order to sense contact of the sealing membrane with the skin of the user.

6. The article of PPE of claim 1, further comprising a second sensor, wherein the second sensor detects whether the article of PPE is in active use.

7. The article of PPE of claim 6, wherein the second sensor detects whether the article of PPE is in active use by detecting at least one of: the article of PPE being powered “ON”, or actuation or movement of a component of the article of PPE relative to the remainder of the article of PPE.

8. The article of PPE of claim 7, wherein usage data further comprises the length of time the article of PPE is in active use.

9. The article of PPE of claim 1, wherein the processor uses the usage data to determine necessary servicing or end-of-life of the article of PPE.

10. The article of PPE of claim 9, wherein the article of PPE is a powered device, and wherein the processor disables the device's ability to power “ON” upon determining necessary servicing or end-of-life of the article of PPE.

11. The article of PPE of claim 1, wherein the article of PPE is a powered device, and wherein the processor disables the device's ability to power “ON” if the device is not being worn.

12. An article of personal protective equipment (PPE), comprising:
a first sensor that detects whether the article of PPE is being worn by a user;
an environmental sensor that detects an environmental factor;
a processing module including:
a clock that measures the length of time that the article of PPE is being worn by the user, and
memory for storage of usage data, wherein usage data includes the length of time the article of PPE has been worn by the user; and
a communications module for wirelessly transmitting stored usage data to a device separate from the article of PPE.

13. The article of PPE of claim 12, wherein the environmental sensor detects at least one of the following environmental characteristics: air quality and ambient noise levels.

14. The article of PPE of claim 13, wherein the environmental sensor generates an environmental rating based on the detected environmental characteristics.

15. The article of PPE of claim 14, wherein usage data further includes the environmental rating.

16. The article of PPE of claim 12, wherein the article of PPE is one of: a mask, a respirator, a hard hat, a welding helmet, protective hearing muffs, an eye protector, protective clothing, protective footwear or a fall protection harness.

17. The article of PPE of claim 12, wherein the first sensor is one of: an accelerometer, a capacitive sensor, a capacitive touch switch, a pressure switch, an acoustic sensor, a reed switch, a temperature sensor, a pressure sensitive switch, a gas sensor, a flow sensor and an optical sensor.

18. (canceled)

19. The article of PPE of claim 12, further comprising a sealing membrane in contact with the skin of the user when the user is wearing the article of PPE, wherein the sensor is disposed in the sealing membrane in order to sense contact of the sealing membrane with the skin of the user.

20. The article of PPE of claim 12, further comprising a second sensor, wherein the second sensor detects whether the article of PPE is in active use.

21. The article of PPE of claim 20, wherein the second sensor detects whether the article of PPE is in active use by detecting at least one of: the article of PPE being powered “ON”, or actuation or movement of a component of the article of PPE relative to the remainder of the article of PPE.

22. The article of PPE of claim 21, wherein usage data further comprises the length of time the article of PPE is in active use.

23. The article of PPE of claim 12, wherein the processor uses the usage data to determine necessary servicing or end-of-life of the article of PPE.

24. The article of PPE of claim 23, wherein the article of PPE is a powered device, and wherein the processor disables the device's ability to power “ON” upon determining necessary servicing or end-of-life of the article of PPE.

25. (canceled)
